(19) United States
(12) Patent Application Publication
(10) Pub. No.: US 2016/0005186 A1
(43) Pub. Date: Jan. 7, 2016

(54) DETERMINATION OF MYOCARDIAL SCAR USING VELOCITY SPECTRAL MAP

(71) Applicant: Quynh A. TRUONG, (US)

(72) Inventor: Quynh A. Truong, Boston, MA (US)

(21) Appl. No.: 14/772,687

(22) PCT Filed: Feb. 18, 2014
(86) PCT No.: PCT/US14/16842

§ 371 Date: Sep. 3, 2015

Related U.S. Application Data
(60) Provisional application No. 61/773,500, filed on Mar. 6, 2013.

(51) Int. Cl. G06T 7/20; A61B 6/00; G06T 11/00; G06T 7/00; G06K 9/46; A61B 6/03; A61B 5/107

(52) U.S. Cl. CPC G06T 7/2033; A61B 6/032; A61B 6/503; A61B 5/1075; G06T 7/0012; G06K 9/46; G06K 9/4604; G06T 7/0028; G06T 11/003; G06T 2207/30048; G06K 2009/4666; G06T 2207/10081; G06T 2200/04

(57) ABSTRACT

A system and method for determining regions of a heart affected by a scar. An imaging dataset is acquired of a portion of a subject including the heart and the imaging dataset is processed to identify a motion parameter of the heart. The motion parameter of the heart is mapped on a spectral scale based on the motion parameter over time to identify low velocity regions of the heart. A report is generated indicating the low velocity regions of the heart affected by scar.

### CROSS-REFERENCE TO RELATED APPLICATIONS

The present application is based on, claims priority to, and incorporates herein by reference in its entirety, U.S. Provisional Application Ser. No. 61/773,500, filed Mar. 6, 2013, and entitled “SYSTEM AND METHOD FOR NON-INVASIVE DETERMINATION OF MYOCARDIAL SCAR USING VELOCITY SPECTRAL MAP.”

### STATEMENT REGARDING FEDERALLY SPONSORED RESEARCH

N/A

### BACKGROUND OF THE INVENTION

The present invention relates to systems and method for analyzing cardiac function and structure. More particularly, the invention relates to a method for determining low velocity heart regions representing myocardial scar of a subject's heart using a spectral scale applied to a set of images based on a motion parameter.

Heart failure is one of the leading causes of morbidity and mortality in the United States. The main therapeutic goals to combat heart failure are focused on reducing major adverse cardiac events (MACE) and mortality, alleviating symptoms and improving functional status. However, despite substantial poly-pharmacological therapies, many patients experience refractory heart failure symptoms. Cardiac resynchronization therapy (CRT) is a device therapy that has gained worldwide acceptance as adjuvant treatment for patients with refractory heart failure, left ventricular (LV) systolic dysfunction, and wide QRS duration. CRT systems build upon pacing systems to also synchronize the function of the heart. Specifically, in addition to pacing the right ventricle, an extra LV lead is typically placed via the coronary sinus into a branch of the coronary veins to pace the LV and “synchronize” the heart. Thus, CRT therapies require invasive deployment of the CRT system.

Unfortunately, about 1 out of 3 patients who receive CRT therapy do not demonstrate clinical improvement. Factors leading to high non-response rate include, for example, suboptimal LV lead placement, intraventricular dyssynchrony, and myocardial scar. Intraventricular (or LV) dyssynchrony occurs when there is delayed electromechanical activation within regions of the left ventricle that result in discordant and inefficient contraction. Given that CRT is expensive, invasive, and carries a procedural risk, improvement of CRT by optimal device implantation is warranted. It is postulated that pacing over the site with maximal discordance and avoiding a region of myocardial scar may result in a better outcome. Additionally, proper patient selection for CRT by examining the extent of dyssynchrony and/or myocardial scar is desirable to reduce the number of unbeneficial implants and provide patients with realistic expectations.

Despite these indications, currently, the vast majority of CRT implantation is performed without pre-procedural imaging of the coronary venous anatomy. At the time of CRT implantation, the LV lead is placed into one of the coronary veins under fluoroscopic guidance. This is performed irrespective of the patient's anatomy and without knowledge of the site of most delayed activation or myocardial scar. If there is not a good vein to target lead placement, then epicardial pacing may be an alternative approach that is used to remediate the failed primary deployment strategy. Thus, pre-operative information on coronary venous anatomy by noninvasive means may be useful in determining the implantation strategy. To provide end-stage heart failure patients with a chance for clinical improvement and to reduce excessive burden and cost on an individual and societal level, a personalized approach to CRT is necessary. Specifically, the LV lead placement should be optimized to target the site of latest activation and avoid areas of myocardial scar.

Imaging of the coronary veins by cardiac computed tomography (CT) prior to CRT implantation is feasible and deemed as “appropriate” in many clinical settings. Neither echocardiography nor nuclear cardiology scans have adequate spatial resolution to allow for coronary venous assessment. Unfortunately, CT imaging, while well suited to anatomical imaging and providing physiological information from anatomical images acquired over time, is currently ill-suited to provide physiological information. In contrast, electroanatomical mapping (EAM) allows measurements of both myocardial electrical activation and myocardial voltage. As the heart is electrically activated (QRS complex), the activation of the LV myocardium starts at the level of the septum, spreading to the apex and then to the base of the heart. With EAM, a color-coded activation map can illustrate areas of delayed activation over time. However, unlike CT imaging, EAM unfortunately is highly invasive. This catheter-based approach also enables the assessment of myocardial voltage map, whereby low voltage areas less than 1.5 mV represent scar. Hence, myocardial scar can be differentiated from non-scarred myocardium by voltage maps at areas of delayed activation on EAM. Both maps are performed with simultaneous intracardiac and surface electrocardiography (ECG). However, EAM is not clinically indicated or performed to guide CRT implantation due to its invasiveness and prolongation of device implantation time.

Therefore, there is a need for systems and methods to non-invasively provide salient information that can be used to assist in planning and implementing CRT procedures to improve clinical outcomes as well as to determine candidates that will receive substantial benefits from CRT from those that will not.

### SUMMARY OF THE INVENTION

The present invention overcomes the aforementioned drawbacks by providing a system and method that noninvasively determines myocardial scar of a subject's heart based on low velocity heart regions to, for example, guide LV lead placement for device therapy. In particular, the present invention can use a spectral scale applied to a set of images to identify low velocity heart regions affected by scar and other information about a subject that can be used to plan or improve implementation of procedures, such as CRT deployment.

In accordance with one aspect of the invention, a system for determining regions of a heart affected by a scar of a subject is disclosed. The system includes a memory having stored thereon an imaging dataset acquired from a portion of the subject including the heart. The system further includes a processor having access to the memory and the imaging dataset stored thereon and configured to process the imaging dataset to identify a motion parameter and map a spectral scale based on the motion parameter over time to identify low velocity regions of the heart. A display is coupled to the processor and configured to display the low velocity regions of the heart relative to a series of images.

In accordance with another aspect of the invention, a method for determining regions of a heart affected by a scar is disclosed. The method includes acquiring an imaging dataset of a subject including the heart and reconstructing the imaging dataset into a set of images. The set of images is then processed to apply a spectral scale based on a motion parameter in order to identify low velocity regions of the heart within the set of images based on the motion parameter. A wall thickness of the heart is determined within the set of images at each low velocity region of the heart and the low velocity regions of the heart having wall thicknesses below a pre-determined threshold are determined. A report is then generated that indicates the low velocity regions of the heart below the pre-determined threshold as being affected by a scar.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of a system configured to implement the present invention.

FIG. 2 is a flow chart setting forth the steps of processes for creating a velocity spectral map in accordance with the present invention.

FIG. 3 is volume rendered velocity maps of a set of images showing a range from low velocity to high velocity heart regions during a cardiac cycle.

### DETAILED DESCRIPTION OF THE INVENTION

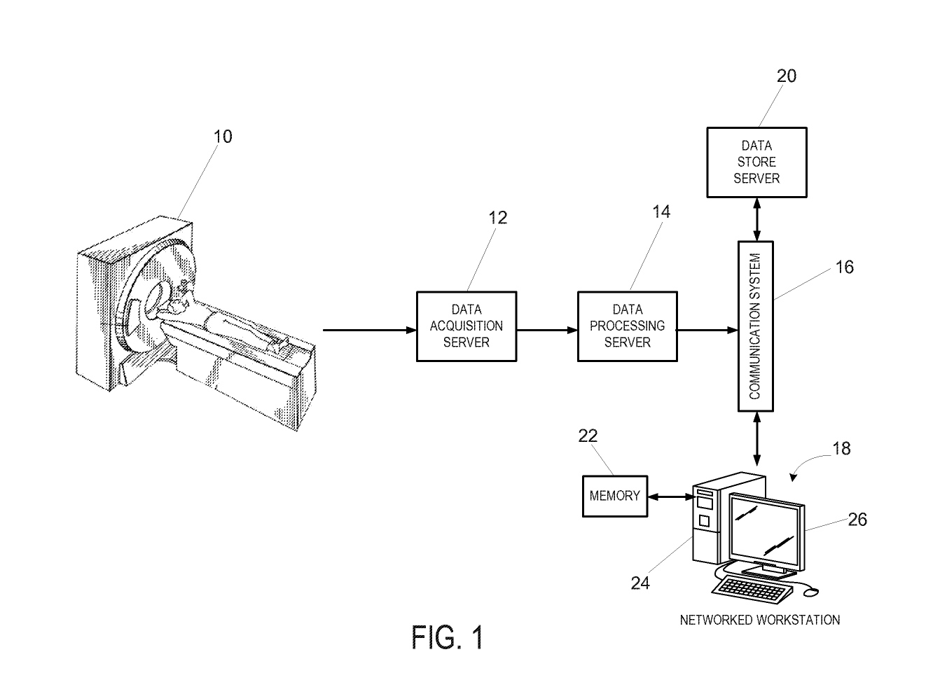

Referring particularly now to FIG. 1, a system 10 is shown that is configured to acquire a raw imaging data from a subject being imaged. The raw data may be, for example, computed tomography (CT) data acquired by a CT imaging system, such as illustrated in FIG. 1; however, other imaging modalities may also be used to acquire the imaging data, such as magnetic resonance imaging (MRI) systems and other imaging systems. For exemplary purposes, and to be consistent with the example system that is illustrated in FIG. 1, reference will be made to a CT dataset; however, other dataset types may be likewise utilized. The raw data is sent to a data acquisition server 12 coupled to the system 10. The data acquisition server 12 then converts the raw data to a dataset suitable for processing by a data processing server 14, for example, to reconstruct one or more images from the dataset. The dataset or processed data or images can then be sent over a communications system 16 to a networked workstation 18 for processing or analysis and/or to a data store server 20 for long-term storage. The communication system 16, which may be local or wide, a wired or wireless, network including, for example, the internet, allows the networked workstation 18 to access the data store server 20, the data processing server 14, or other sources of information.

The networked workstation 18 includes a memory 22 that can store information, such as the dataset. The networked workstation 18 also includes a processor 24 configured to access the memory 22 to receive the dataset or other information. The network workstation 18 also includes a user communication device, such as a display 26, that is coupled to the processor 24 to communicate reports, images, or other information to a user.

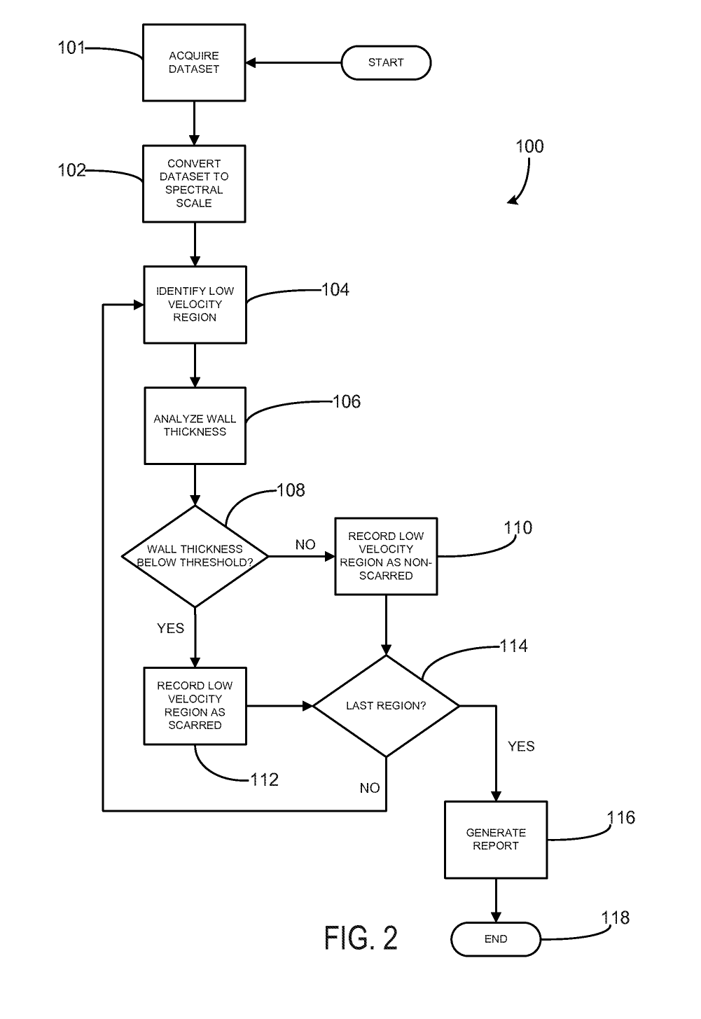

Referring now to FIG. 2, a flow chart setting forth exemplary steps 100 for creating a velocity spectral map to derive myocardial scar information about a subject non-invasively is provided. To start the process a dataset is obtained at process block 101. The dataset, for example, may be a CT dataset acquired with a CT system, or other imaging or dataset about the subject's heart. In the example of a CT dataset, a clinician may operate a CT imaging system, such as the system 10 of FIG. 1, to acquire a CT dataset specifically for this process, or a previously-acquired CT dataset that includes a portion of the patient's heart may be accessed. Regardless of the imaging modality, the dataset includes information about the cardiac anatomy and motion of the cardiac anatomy over time. The dataset may include previously-reconstructed images or raw imaging data that is then reconstructed.

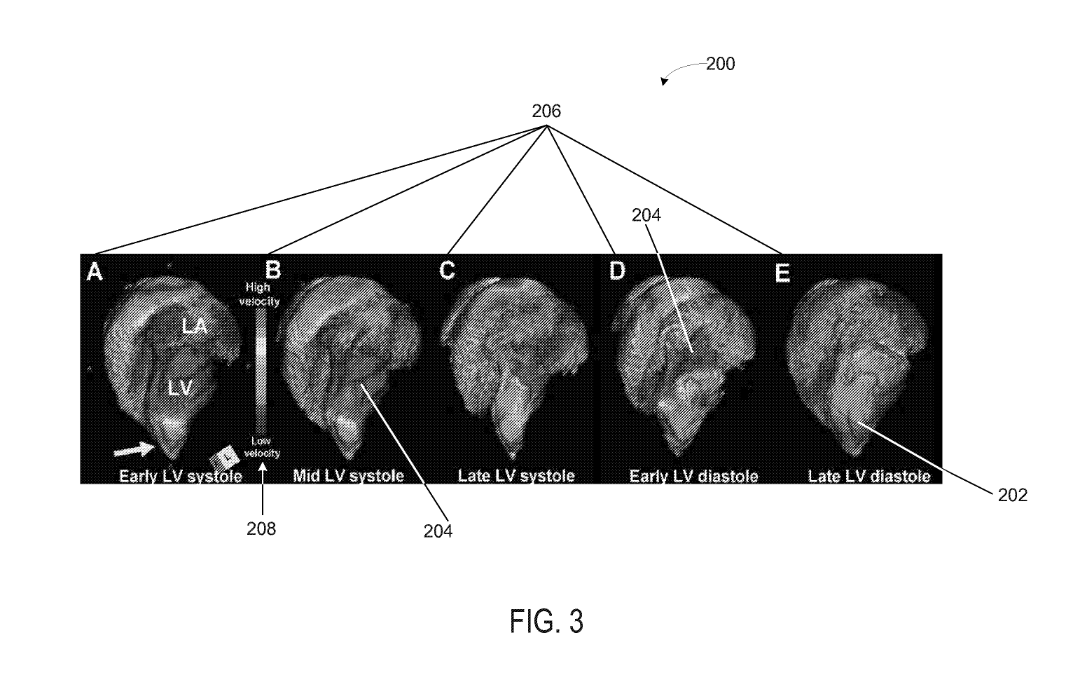

The dataset, preferably after reconstruction into images, is then converted to a spectral scale at process block 102. The spectral scale can be a non-binary grayscale template 208, as shown in FIG. 3, and created based on a motion parameter, for example, a velocity parameter. Alternatively, the spectral scale can be a non-binary color template also created based on a motion parameter, such as a velocity parameter. That is, to convert the dataset to a spectral scale, the dataset, for example, as reconstructed images, is analyzed using a motion parameter, such as velocity. In one example, the anatomical images, as described above, may be processed at, for example, five percent increments of the R-R interval (20 phases) to identify the beginning of the R-R interval and, using these images, the cardiac phases 206, as shown in FIG. 3, are identified. For example, the end of the systole and diastole cardiac phases of the cardiac cycle may be identified and tagged within the images.

Further, the motion may be processed to determine and track a predetermined motion parameter over time. For example, the processing of the motion dataset, in one example, may include using a non-rigid registration (60 phases) algorithm and tracking voxel-to-voxel movement throughout the cardiac cycle in order to yield an estimated velocity and acceleration which can be tracked throughout ventricular systole and diastole. The non-rigid registration algorithm may be designed to align the cardiac structures from phase-to-phase, virtually tracking individual voxels through the cardiac cycle. Further, by applying physics modeling, parameters such as velocity can be calculated, such that during cardiac contraction and relaxation, myocardial velocity depicts the distance the myocardium has moved over time (mm/sec) and acceleration as velocity over time (mm/sect). Once the processing of the motion is complete, a motion parameter, for example, velocity, is identified to operate as a surrogate for electro-mechanical activation. For example, a time-to-first-peak systolic velocity may be identified.

Once the cardiac phases and motion parameter operating as a surrogate for electro-mechanical activation are identified, the cardiac anatomy data (e.g., images) and motion dataset (e.g., tracked motion parameters) are merged. In doing so, the anatomic dataset may be used to segregate the motion dataset to localize the motion dataset to the heart by superimposing the deformable color kinematic/velocity maps, for example, over the cardiac anatomy datasets.

Once the dataset is converted to a spectral scale at process block 102 as described above, low velocity heart regions can be identified at process block 104. Examples of low velocity heart regions 202 are shown in FIG. 3, as well as high velocity heart regions 204. Low velocity regions that remain low velocity throughout systole and diastole may be considered scarred myocardium. Regions of non-scarred myocardium may be low velocity during a particular time of the cardiac cycle, but then change to higher velocity values at other times of the cardiac cycle, such as indicated by region 202.

The wall thickness of the myocardium may be analyzed at process block 106 once the low velocity heart regions are identified at process block 104. For example, the wall thickness can be measured by the boundaries of the endocardium and the epicardium, which can be automatically identified by the processor 24 as shown in FIG. 1 using the anatomical images. The wall thicknesses are then compared to a predetermined threshold at process block 108. If the myocardium wall thicknesses are below the predetermined threshold at process block 108, they are recorded as scarred low velocity heart regions at process block 112. For example, a myocardium wall thickness of 6mm or less that does not change in between systole and diastole is recorded as scarred myocardium. However, if the myocardium wall thicknesses are above the predetermined threshold at process block 108, they are recorded as non-scarred, low velocity heart regions at process block 110. In other words, a constant low velocity throughout the cardiac cycle, for example, at heart regions below the predetermined wall thickness threshold correspond to regions of myocardial scar, whereas a variable low velocity value at heart regions above the predetermined wall thickness threshold correspond to non-scarred, but impaired myocardium. Thus, identifying low velocity heart regions below the predetermined wall thickness threshold allows clinicians to identify optimal placement of an LV lead in viable myocardium and avoid regions of myocardial scar.

At decision block 114, a check is made to determine whether there are any remaining wall thicknesses of low velocity heart regions to be recorded. If there are additional heart regions with wall thicknesses that need to be identified, and, if so, the preceding process flow is repeated from process block 104. If there are not additional heart regions with wall thicknesses that need to be identified at decision block 114, a report is generated at process block 116.

The report generated at process block 116 is based on the preceding analysis. For example, three-dimensional (3D) volume-rendered images of the coronary veins fused over a parametric map, for example a color-encoded parametric map, that identifies regions of myocardial scar and the region of most delayed activation throughout the cardiac cycle may be created, as shown in FIG. 3. The report may identify regions of myocardial scar and the region of most delayed activation. The generated report can include images and/or videos of movement of myocardial segments throughout the cardiac cycle displayed as parametric maps overlaying volume rendered images, such that patterns of myocardial activation conduct to similar patterns of mechanical motion due to effects of electromechanical coupling. Electromechanical coupling is a process that links electrical cardiac excitation to mechanical contraction of the myocardium. Therefore, areas of ischemic or scarred myocardium with impaired kinematics 202 (e.g., low velocity) may be represented on one end of the spectral scale 208 as compared to normal myocardium 204 (e.g., high velocity) in the generated report.

Additionally, the above-described process can be used to map contractility to reflect the activation pattern seen on electroanatomical map (EAM) in order to identify the site of latest activation to guide left ventricular lead placement with device therapy for pre-procedural planning. Thus, the spectral functional velocity map 200 shown in FIG. 3, which may be color-encoded for example, can identify low velocity heart regions 202 on one end of the spectral scale 208 and high velocity heart regions 204 on another end of the spectral scale 208 during phases of the cardiac cycle 206. Further, as shown in FIG. 3, the left ventricular myocardium is isolated and only the velocity that corresponds to myocardium is included, such that the velocity visualized represents myocardial velocity and not its adjacent structures. Once the report is generated at process block 116 of FIG. 2, the steps 100 for creating a velocity spectral map to derive myocardial scar information about a subject non-invasively is completed at process block 118.

The above-described process, as a non-limiting example, may be embodied as post-processing kinematics software to assess myocardial velocity and acceleration, thereby providing a non-invasive imaging tool with potential widespread clinical applications. The strength of cardiac CT lies in the ability to clearly demonstrate anatomy, however at present, evaluation of myocardial function is limited to gray-scale images. Myocardial velocity and acceleration measured on along the longitudinal, radial and circumferential directions of the LV provides the ability to quantitatively assess, for example, LV global and regional myocardial contractility. Moreover, integration of cardiac anatomy with function by superimposing velocity color maps onto, for example, gray scale CT images provides previously-unavailable information to clinicians without the need for interventional procedures.

Thus, the above-described system and method used allows a clinician to use CT-acquired data to non-invasively determine the optimal site of LV lead placement for CRT by the co-registration of anatomic (e.g., coronary veins) and functional (e.g., LV dyssynchrony and myocardial scar) data to avoid regions of myocardial scar and to target regions of most delayed activation. The present invention provides functional analytics that provide a non-invasive way to quantitatively measure regional myocardial velocity and acceleration.

The present invention has been described in terms of one or more preferred embodiments, and it should be appreciated that many equivalents, alternatives, variations, and modifications, aside from those expressly stated, are possible and within the scope of the invention.

1. A system for determining regions of a heart affected by a scar, the system comprising:
a memory having stored thereon an imaging dataset acquired from a portion of a subject including the heart;
a processor having access to the memory and the imaging dataset stored thereon and configured to process the imaging dataset to identify a motion parameter and map a spectral scale based on the motion parameter over time to identify low velocity regions of the heart; and
a display coupled to the processor and configured to display the low velocity regions of the heart relative to a series of images.

2. The system as recited in claim 1 wherein the processor is further configured to determine a wall thickness of the heart within the series of images at each low velocity region of the heart.

3. The system as recited in claim 2 wherein the processor is further configured to determine the low velocity regions of the heart within the series of images having the wall thicknesses below a pre-determined threshold.

4. The system as recited in claim 2 wherein the wall thickness is defined with respect to boundaries of a endocardium and a epicardium.

5. The system as recited in claim 4 wherein the processor is further configured to analyze the boundaries of the endocardium and the epicardium throughout a cardiac cycle of the low velocity regions of the heart.

6. The system as recited in claim 1 wherein the motion parameter includes velocity and the processor is configured to determine the velocity using a non-rigid registration based algorithm to track a voxel-to-voxel movement during a cardiac cycle to indentify the low velocity regions of the heart.

7. The system as recited in claim 6 wherein the voxel-to-voxel movement is expressed as velocity.

8. The system as recited in claim 1 wherein the display shows at least one of a series of images and a video.

9. The system as recited in claim 1 wherein the processor is further configured to identify scarred myocardium on a first end of the spectral scale and identify non-scarred myocardium on a second end of the spectral scale to be displayed by the display relative to the series of images.

10. The system as recited in claim 1 wherein the processor is further configured to identify a site of non-scarred myocardium for left ventricular lead placement.

11. The system as recited in claim 1 wherein the imaging dataset is a CT dataset.

12. A method for determining regions of a heart affected by a scar, the method comprising the steps of:
a) acquiring an imaging dataset of a subject including the heart;
b) reconstructing the imaging dataset into a set of images;
c) processing the set of images to apply a spectral scale based on a motion parameter;
c) identifying low velocity regions of the heart within the set of images based on the motion parameter;
d) determining a wall thickness of the heart within the set of images at each low velocity region of the heart;
e) determining low velocity regions of the heart within the set of images having wall thicknesses below a pre-determined threshold; and
f) generating a report indicating the low velocity regions of the heart below the pre-determined threshold as being affected by a scar.

13. The method as recited in claim 12 wherein the wall thickness of step d) is defined as boundaries of an endocardium and an epicardium.

14. The method as recited in claim 13 further comprising the step of analyzing the boundaries of the endocardium and the epicardium throughout a cardiac cycle of the low velocity regions of the heart.

15. The method as recited in claim 12 wherein step c) includes further processing a velocity dataset using a non-rigid registration based algorithm to track a voxel-to-voxel movement during a cardiac cycle.

16. The method as recited in claim 15 further comprising the step of expressing the voxel-to-voxel as velocity when the motion parameter includes velocity.

17. The method as recited in claim 12 wherein generating the report includes generating at least one of a series of images and a video.

18. The method as recited in claim 12 further comprising the steps of identifying scarred myocardium on a first end of the spectral scale in the repot and identifying non-scarred myocardium on a second end of the spectral scale in the report.

19. The method as recited in claim 12 further comprising the step of identifying a site of non-scarred myocardium for left ventricular lead placement in the report.

20. The method as recited in claim 12 wherein acquiring an image dataset includes acquiring a CT dataset.
